SEQUENCE LISTING (20 sequences)

SEQ ID NO: 1 - protein, 46 aa, Artificial Sequence
  Synthetic
VDEQIELTTAEPYAPPTLTEVGEFNEDTLGFIGWGNDIFGHYSGGF

SEQ ID NO: 2 - protein, 35 aa, Artificial Sequence
  Synthetic
MAYERPTLTKVGDFQKVTGLAGQGSPDLLGGHSLL

SEQ ID NO: 3 - protein, 36 aa, Artificial Sequence
  Synthetic
MKKAYEAPTLVRLGSFRKQTGLLGLAGNDRLVLSKN

SEQ ID NO: 4 - protein, 44 aa, Artificial Sequence
  Synthetic
MTESIEAYEPPMLVEVGSFAELTRSYHWGDYHDWHHGWYGWWDD

SEQ ID NO: 5 - protein, 46 aa, Artificial Sequence
  Synthetic
MDEEKIGNEISAYETPTVTEMGAFSEVTLGRPNWGFENDWSCVRVC

SEQ ID NO: 6 - protein, 17 aa, Artificial Sequence
  Synthetic
GRPNWGFENDWSCVRVC

SEQ ID NO: 7 - protein, 40 aa, Artificial Sequence
  Synthetic
MEKKKYTAPQLAKVGEFKEATGWYTAEWGLELIFVFPRFI

SEQ ID NO: 8 - protein, 45 aa, Artificial Sequence
  Synthetic
MQEKKTQRKKYTRPQIAKAGGFKESTGWIQGKWGLEIYLIFPRYL

SEQ ID NO: 9 - protein, 6 aa, Artificial Sequence
  Synthetic
  MISC_FEATURE (2)..(2): Xaa can be any residue
  MISC_FEATURE (3)..(3): Xaa can be any residue
  MISC_FEATURE (5)..(5): Xaa can be any residue
YXXPXL

SEQ ID NO: 10 - protein, 27 aa, Artificial Sequence
  Synthetic
MEKQKETKKEYSSPRLIELGDIVEITF

SEQ ID NO: 11 - protein, 21 aa, Artificial Sequence
  Synthetic
GGKPGWGSDTYSQRYPRSDED

SEQ ID NO: 12 - protein, 22 aa, Artificial Sequence
  Synthetic
MEKKKYTAPQLAKVGEFKEATG

SEQ ID NO: 13 - protein, 18 aa, Artificial Sequence
  Synthetic
WYTAEWGLELIFVFPRFI

SEQ ID NO: 14 - DNA, 50 bp, Artificial Sequence
  Synthetic
atggcagatctcaattggatatcaatggagacaacaggagctgaattccg

SEQ ID NO: 15 - DNA, 54 bp, Artificial Sequence
  Synthetic
gctcatatgtatatctccttcttatacttaactaatcatggcagcgccatcccg

SEQ ID NO: 16 - DNA, 51 bp, Artificial Sequence
  Synthetic
ttagttaagtataagaaggagatatacatatgagcgagaacgtagtgctgc

SEQ ID NO: 17 - DNA, 44 bp, Artificial Sequence
  Synthetic
tttaccagactcgagggtacctcagtccacggtgatcagacggc

SEQ ID NO: 18 - DNA, 34 bp, Artificial Sequence
  Synthetic
aaaagtcgacatggtcggttgcatcagtccttac

SEQ ID NO: 19 - DNA, 33 bp, Artificial Sequence
  Synthetic
aaagcggccgctcagctcctgttgtctccaccg

SEQ ID NO: 20 - protein, 18 aa, Artificial Sequence
  Synthetic
  MISC_FEATURE (1)..(1): Xaa can be W, H, K, L, or A.
XYTAEWGLELIFVFPRFI

(19) United States
(12) Patent Application Publication
(10) Pub. No.: US 2021/0108191 A1
(43) Pub. Date: Apr. 15, 2021

(54) Methods of Production of Biologically Active Lasso Peptides

(71) Applicant: The Board of Trustees of the University of Illinois, Urbana, IL (US)

(72) Inventor: Douglas Alan MITCHELL, Urbana, IL (US)

(73) Assignee: The Board of Trustees of the University of Illinois, Urbana, IL (US)

(21) Appl. No.: 16/500,623

(22) PCT Filed: Apr. 4, 2018
(86) PCT No.: PCT/US2018/026101

§ 371 Date: Oct. 3, 2019

Related U.S. Application Data
(60) Provisional application No. 62/481,677, filed on Apr. 4, 2017.

(51) Int. Cl. C12N 15/10; C07K 14/195; C12N 15/63

(52) U.S. Cl. CPC C12N 15/1037; C12N 15/63; C07K 14/195

(57) ABSTRACT

Recombinant and in vitro reconstitution methods for producing lasso peptides are provided. Methods of screening lasso peptides are also provided.

### PRIORITY

This application claims the benefit of U.S. Ser. No. 62/481,677, filed on Apr. 4, 2017, which is incorporated by reference herein in its entirety.

### BACKGROUND

Lasso peptides are ribosomally assembled and post-translationally modified peptides (RiPPs) that are produced by bacteria. Lasso peptides are some of the smallest possible globular proteins (FIG. 1A). However, the unique topology renders the fold inaccessible to chemical synthesis but remarkably stable towards heat and proteases. Combined with their naturally diverse and expansive surfaces relative to small molecules, lasso peptides are an ideal starting point to inhibit (or associate) targets deemed undruggable by small molecules.

Lasso peptides, however, are difficult to recombinantly or synthetically produce. Thermophilic and actinomycete mature lasso peptides and lasso peptides of the biosynthetic gene cluster are particularly difficult to produce in the laboratory. Methods are needed in the art to produce biologically active, soluble, and stable mature lasso peptides and biologically active, soluble, and stable lasso peptides of the synthetic gene cluster.

### SUMMARY

An embodiment provides methods of producing a mature lasso peptide. The methods comprise transforming a host cell with a first plasmid comprising a nucleic acid molecule encoding a lasso precursor peptide operably linked to a solubility enhancing polypeptide and a second plasmid comprising a nucleic acid molecule encoding a lasso leader peptidase; a nucleic acid molecule encoding a lasso cyclase; and a nucleic acid molecule encoding a RiPP recognition element (RRE) to generate a transformed host cell. The transformed host cell can be cultured in media. The mature lasso peptide can be extracted from the host cell or the culture media. The lasso peptide can be produced at a yield of more than 0.5 mg/L of culture media. The nucleic acid molecule encoding a lasso precursor peptide can be an actinomycete or thermophile nucleic acid molecule. The nucleic acid molecule encoding a lasso leader peptidase can be an actinomycete or thermophile nucleic acid molecule. The nucleic acid molecule encoding a lasso cyclase can be an actinomycete or thermophile nucleic acid molecule. The nucleic acid molecule encoding a RiPP recognition element (RRE) can be an actinomycete or thermophile nucleic acid molecule. Various combinations of actinomycete or thermophile nucleic acid molecules can be used. The host cell can be a mesophile. The mature lasso peptide can be an actinomycete lasso peptide or a thermophile lasso peptide.

Another embodiment provides methods of producing one or more peptides of a lasso biosynthetic gene cluster. The methods can comprise transforming a host cell with (i) a plasmid comprising a nucleic acid molecule encoding an actinomycete lasso cyclase or a thermophile lasso cyclase operably linked to a solubility enhancing polypeptide; (ii) a plasmid comprising a nucleic acid molecule encoding a thermophile lasso leader peptidase or an actinomycete lasso leader peptidase operably linked to a solubility enhancing polypeptide; (iii) a plasmid comprising a nucleic acid molecule encoding a thermophile lasso RiPP recognition element or an actinomycete lasso RiPP recognition element operably linked to a solubility enhancing polypeptide; (iv) a plasmid comprising a nucleic acid molecule encoding a thermophile lasso precursor peptide or a operably linked a solubility enhancing polypeptide; or (v) a combination thereof; to generate a transformed host cell. The transformed host cell can be cultured in a culture media. The one or more peptides of a lasso biosynthetic gene cluster can be extracted from the host cell or the culture media. The host cell can be a mesophile.

Still another embodiment provides methods of producing a mature lasso peptide in vitro comprising combining one or more purified recombinant actinomycete or thermophile lasso cyclases, one or more purified recombinant actinomycete or thermophile lasso leader peptidases, one or more purified recombinant actinomycete or thermophile lasso RiPP recognition elements, and one or more purified recombinant actinomycete or thermophile lasso precursor peptides) in vitro under conditions suitable for lasso peptide formation, such that a mature lasso peptide is produced. The lasso peptide can be produced at a yield of more than 1 mg/L.

Another embodiment provides a method of producing a mature lasso peptide in vitro comprising combining one or more purified recombinant lasso precursor peptides lacking a leader portion, one or more purified recombinant lasso cyclases, and adenosine triphosphate (ATP) in vitro under conditions suitable for lasso peptide formation; such that a mature lasso peptide is produced. The lasso precursor peptide lacking a leader portion can be an actinomycete or thermophile peptide. The lasso cyclase can be an actinomycete or thermophile peptide.

Yet another embodiment provides methods of screening for biological activity of a lasso peptide. The method comprises displaying one or more tagged lasso precursor peptides in a display library. The display library is contacted with a purified lasso cyclase, a purified lasso leader peptidase, and a purified lasso RiPP recognition element to form a lasso peptide display library. The lasso peptide display library can be with one or more test agents. The display library can be a phage display library, a phagemid display library, a virus display library, a bacterial cell display library, yeast display library, a λgt11 library, an in vitro library selection system (CIS display), an in vitro compartmentalization library, an antibody-ribosome-mRNA (ARM) ribosome display library, or a ribosome display library. The one or more tagged lasso precursor peptides can be mesophile, actinomycete, or thermophile peptides. The lasso cyclase, the lasso leader peptidase, and the lasso RiPP recognition element can be actinomycete or thermophile peptides.

Another embodiment provides a display library comprising a plurality of displayed mature lasso peptides. The display library can be a phage display library, a phagemid display library, a virus display library, a bacterial cell display library, yeast display library, a λgt11 library, an in vitro library selection system (CIS display), an in vitro compartmentalization library, an antibody-ribosome-mRNA (ARM) ribosome display library, or a ribosome display library. The mature lasso peptides can be actinomycetes mature lasso peptides or thermophile mature lasso peptides.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. Panels A-B. A, Space-filling model of a typical lasso peptide, acinetodin (PDB 5U16), highlighting the globular shape and dimensions, B, Lasso peptide BGC with essential genes highlighted. The gene nomenclature and coloring adopted throughout is: A, precursor peptide (black); 8, leader peptidase (homologous to transglutaminase, orange); C, lasso cyclase (homologous to asparagine synthetase, blue); E, RRE (RiPP Recognition Element, homologous to PqqD, yellow). Precursors are comprised of leader and core regions. After leader removal, the cyclase is presumed to adenylate Asp/Glu. The substrate must be “pre-folded” prior to macrocyclization given the known steric restraints

FIG. 2. RODEO workflow. The RODEO algorithm takes a list of protein accessions as input and retrieves a user-defined number of flanking genes from GenBank. These genes are then functionally analyzed. Lastly; RODEO performs a 6-frame translation of intergenic regions. These peptides are then scored to identify the most likely lasso precursor. See: ripprodeo.org for more information.

FIG. 3. Panels A-E. RODEO-enabled insights into lasso peptides. A, Distribution of BGCs amongst bacteria. B, BGCs of two anantins (previously unknown origin) and four novel lassos. Naming and color-coding identical to FIG. 1. C, Precursor sequences corresponding to panel (B). A previously unrecognized but prevalent YxxPxL (SEQ ID NO:9), and known Gx₅T, leader motifs are red. Core residues found in the macrocycle are blue as well as two modifications (citrulline, orange; disulfide; green). AnaA is SEQ ID NO:1; LagA is SEQ ID NO:2; CitA is SEQ ID NO:3; MooA is SEQ ID NO:4; LpeA is SEQ ID NO:5. D, Topologies of lasso peptides. Frequencies for class I-IV are 1.4%, 96%, 2%, 0.6%, respectively. E, Solution structure of LP2006: a rare disulfide-containing, class IV lasso peptide (SEQ ID NO:6).

FIG. 4. Panels A-E. Thermobifida lasso peptides. A, Sequences of TfusA (SEQ ID NO:7)/TcelA (SEQ ID NO:8)(BGC architecture identical to FIG. 3B), B, MS-confirmed E. coli production of fusilassin. C, Simplified structural representation of fusilassin. D, SDS-PAGE of MBP-tagged Tcel and Tfus proteins (unoptimized expression, single-column purification). Yields for TcelABCE and TfusABCE, respectively: 5, 14, 8, 50 mg/L and 8, 20, 3, 32 mg/L. E, MALDI-TOF-MS data showing in vitro reconstitution of fusilassin biosynthesis (m/z 2269). Omission of ATP or TfusC yields cleaved core (m/z 2287). Omission of TfusB or TfusE gives no reaction (unmodified precursor m/z >5000).

FIG. 5 shows the structure of pACYC-TfusC-TFusEB.

FIG. 6 shows heterologous expression/biosynthesis of fusilassin in E. coli.

FIG. 7 shows a general structure of a lasso peptide.

FIG. 8 shows an example of a two plasmid construct for expression of a lasso peptide by a host cell.

FIG. 9 shows an alternate construct for expression of a lasso peptide by a host cell.

FIG. 10 shows an alternate construct for expression of a lasso peptide by a host cell.

FIG. 11 shows MALDI-TOF-MS data showing in vivo production of fusilassin biosynthesis (m/z 2269) and in vivo production of position 1 mutants of fusilassin. Position 1 of the core peptide was substituted with W (m/z 2269), H (m/z 2220), K (m/z 2211), L (m/z 2196), or A (m/z 2154). XYTAEWGLELIFVFPRFI is SEQ ID NO:20.

### DETAILED DESCRIPTION OF THE INVENTION

The compositions and methods are more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

The terms used in the specification generally have their ordinary meanings in the art, within the context of the compositions and methods described herein, and in the specific context where each term is used. Some terms have been more specifically defined below to provide additional guidance to the practitioner regarding the description of the compositions and methods.

### Lasso Peptides

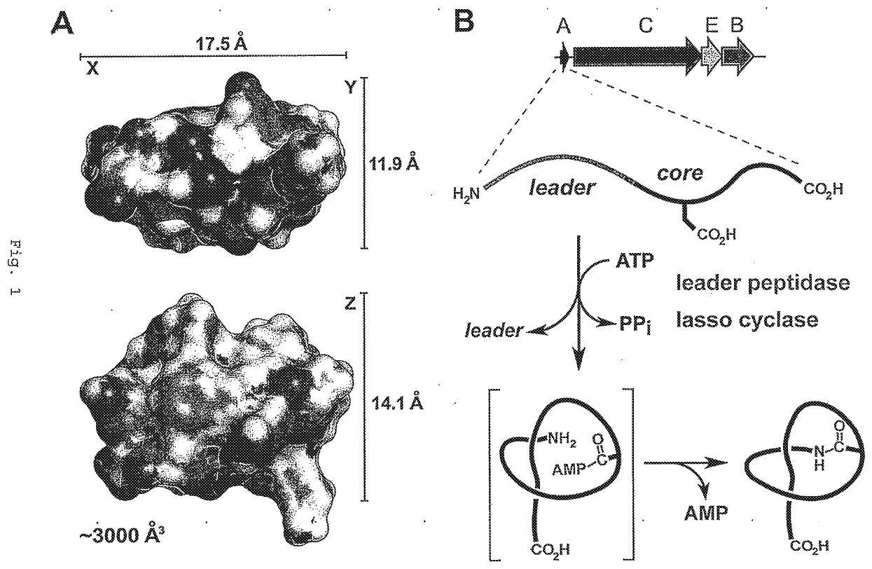

Lasso peptides are ribosomally synthesized and post-translationally modified peptidic (RiPP) products. RiPP biosynthesis begins with the processing of a gene-encoded precursor peptide, which is comprised of functionally distinct leader and core portions (FIG. 1B). The leader harbors a unique motif, known as the recognition sequence, that specifically recruits biosynthetic proteins while the core region receives all of the chemical modifications. After modification, the leader is proteolytically removed. Based on the bipartite nature of RiPP precursors, the biosynthetic enzymes can be specific for a particular recognition sequence yet promiscuously process diverse, even unrelated, core sequences. Furthermore, RiPPs require minimal genomic space, with many lasso peptide biosynthetic gene clusters (BGCs) needing <3 kb.

The lasso topology imparts notable stability. While lasso peptide biosynthesis is known is general terms, there is little molecular insight into how lasso peptides are actually formed. Lasso peptides occupy a chemical and functional space between small molecules and proteins. Indeed, rather than behaving like a polypeptide, the traits of a lasso peptide are more consistent with a small molecule. In support of this observation, lasso peptides have therapeutic activity after oral administration, which conflicts with a long-standing dogma regarding peptidic drugs.

Examples of lasso peptide bioactivities include antagonism of various cell-surface proteins, including the receptors for atrial natriuretic peptide, glucagon, and endothelin. Notably, the lasso peptide siamycin interacts with HIV envelope proteins and prevents viral fusion to CD4⁺ T cells. Enzymes can also be lasso peptide targets, as illustrated by lassomycin, which selectively blocks mycobacterial ClpC1 protease, as well as microcin J25 and capistruin which inhibit RNA polymerase after employing a Trojan horse-like strategy to gain entry into Gram-negative proteobacteria.

Macrocyclic peptides with notable bioactivities include influenza virus fusion inhibitors, PD-1/PD-L1 inhibitors for anticancer applications, insulin-degrading enzyme inhibitors for type-2 diabetes, etc. However, the discovery and refinement of such cyclic peptides are typically achieved only after multiple rounds of chemical synthesis and bioactivity assays, or are reliant on existing libraries of macrocyclic compounds. Lasso peptides recapitulate the desired properties of synthetic cyclic peptides (e.g. siamycin also blocks viral fusion) but with two major advantages: (i) lasso peptides are entirely genetically encoded and (ii) the presence of a free C-terminal tail in the lasso provides an anchor point for surface display or other high-throughput screening methods.

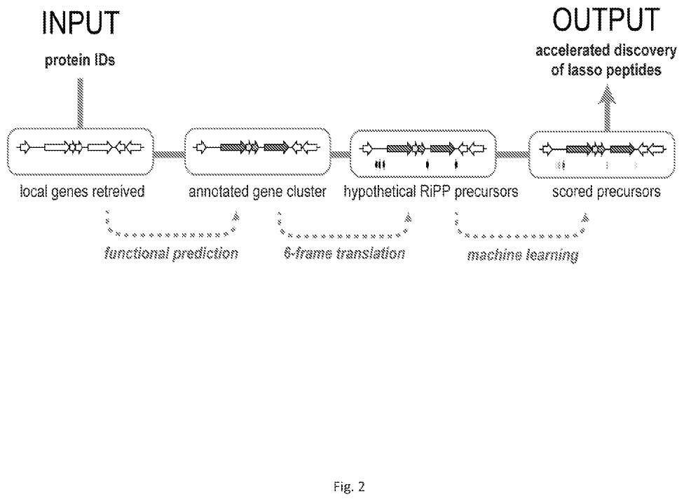

Rapid ORF Description & Evaluation Online (RODEO) can be used to identify lasso gene clusters. See, Tietz et al., A new genome-mining tool redefines the lasso peptide biosynthetic landscape. Nature Chemical Biology, 13:470-478 (2017), which is incorporated herein by references in its entirety. This program profiles genes neighboring a query and automates the genome-mining process (FIG. 2). RODEO is distinguished from other programs (e.g., antiSMASH and PRISM) by taking a protein-centric perspective to genome-mining (i.e. all lasso peptide gene clusters can be queried in a single run rather than one query per genome). RODEO accurately predicts several classes of RiPP precursor peptides.

Lasso peptide biosynthesis remains largely enigmatic. This lack of understanding is a function of the difficulties in identifying lasso peptide gene clusters prior to the development of RODEO and the poor stability/solubility of purified lasso peptide biosynthetic enzymes.

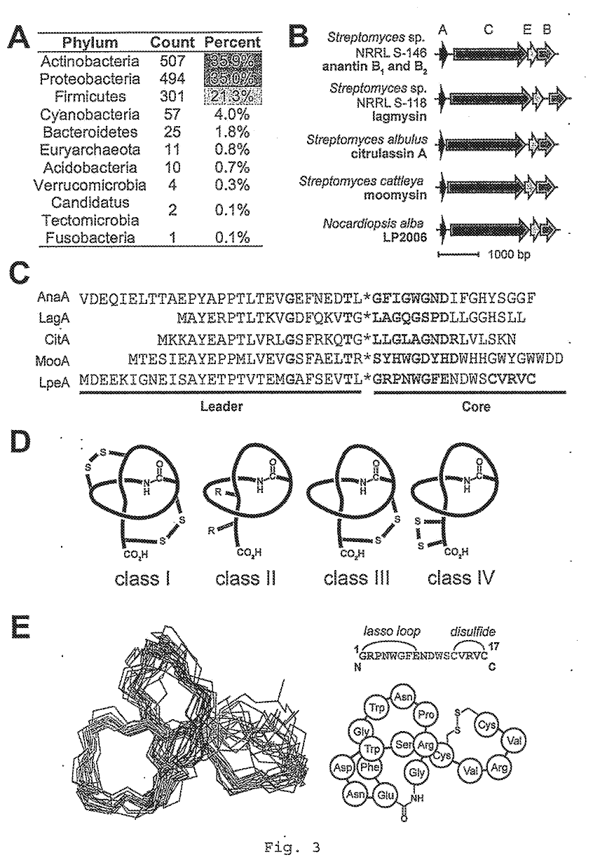

RODEO can be used to assist in identifying lasso peptide gene clusters (FIG. 2). Retrospective analysis of data generated using RODEO revealed unforeseen trends in the phylogenomics, sequence biases, domain architectures, and topologies of lasso peptides (FIG. 3). Several new lasso peptides have been characterized. (FIG. 3B-C). For example, LP2006 displays a previously unprecedented class IV topology (PDB code: 5JPL, FIG. 3D) and was active against several pathogenic bacteria. RODEO provides a roadmap for the discovery of new RiPPs and lasso peptide biosynthetic enzymology.

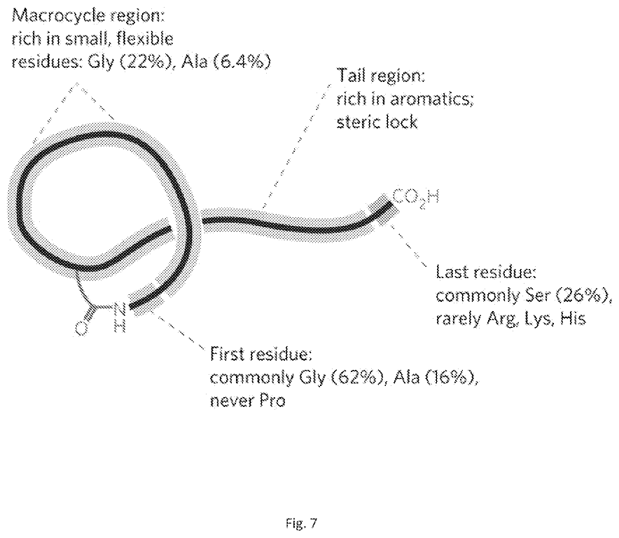

In an embodiment methods of recombinantly producing mature lasso peptides are provided. In an embodiment methods of producing mature lasso peptides via in vitro reconstitution are provided. A mature lasso peptide is a post-translationally modified, biologically active molecule as shown in, e.g., FIGS. 1B, 3D, and 8. Lasso peptides comprise about 20 amino acids, with an average size range of about 15, 16, 17, 18, 19, 20, 21, 22, 23, 24, or 25 amino acids, with molecular weights of about 1500-2500 Da). Lasso peptides comprise a ring of about 7, 8, or 9 amino acids, which can be closed by a lactam bond between the N-terminal amino group and the carboxylate side chain of a glutamate or an aspartate. The tail can be trapped within the ring either by bulky side chains (steric trapping) or by one or two disulfide bonds, or by both means. This specific lasso (or lariat) topology makes lasso peptides extraordinarily stable. See FIG. 7,

### Multiple Plasmid Method of Production of Lasso Peptides

Methods of recombinantly producing mature lasso peptides comprise transforming a host cell with a first plasmid or a first nucleic acid molecule comprising a nucleic acid molecule encoding a lasso precursor peptide operably linked to a solubility enhancing polypeptide and a second plasmid or second nucleic acid molecule comprising a nucleic acid molecule encoding a lasso leader peptidase; a nucleic acid molecule encoding a lasso cyclase; and a nucleic acid molecule encoding a RiPP recognition element (RRE) to generate a transformed host cell.

A solubility enhancing polypeptide or tag is a polypeptide that is operably linked to a protein (e.g., lasso precursor peptide, lasso leader peptidase; lasso cyclase; a RiPP recognition element (RRE)), and which can help to properly fold the protein of interest leading to enhanced solubility of the protein of interest. In an embodiment the concentration, total yield, solubility, biological activity, or combinations thereof are about 5, 10, 15, 20, 25, 30, 40, 50, 60, 75% or more improved for the protein of interest when a solubility enhancing polypeptide or tag is used as compared to the concentration, total yield, solubility, biological activity, or combinations thereof where a solubility enhancing polypeptide is not used. Examples of solubility enhancing polypeptides include maltose binding protein (“MBP”) (Lebendiker & Danieli, Purification of proteins fused to maltose-binding protein, Methods Mol. Biol. 681:281 (2011)), thioredoxin, transcription elongation factor NusA, thiol-disulfide oxidoreductase, glutathione S-transferase (GST), protein G B1 domain, protein D, the Z domain of Staphylococcal protein A, GB1^{basic}, Calmodulin, Poly Arg or Lys peptide tags, SUMO small ubiquitin-modifier, synthetic solubility-enhancing tags, DsbC Disufide bond C, Skp seventeen kilodalton protein, T7PK Phage T7 protein kinase, and ZZ (protein A IgG ZZ repeat domain).

A solubility enhancing polypeptide or tag can be linked or fused to an N-terminus or a C-terminus of the protein of interest.

The transformed host cell is cultured in a culture media such that mature lasso peptides that are post-translationally modified and biologically active are produced. The lasso peptides can be extracted or isolated from the host cell or the culture media.

In an embodiment, the second plasmid (which comprises one or more nucleic acid molecules encoding a lasso leader peptidase; one or more nucleic acid molecules encoding a lasso cyclase; and one or more nucleic acid molecules encoding a RiPP recognition element (RRE)) can be replaced with multiple plasmids. That is one or more of the nucleic acid molecules encoding a lasso leader peptidase; one or more of the nucleic acid molecules encoding a lasso cyclase; and one or more of the nucleic acid molecules encoding a RiPP recognition element (RRE)) can each be present on a single plasmid or a combination of two or more of the nucleic acids can be present on a single plasmid. In an embodiment the one or more nucleic acid molecules encoding a lasso leader peptidase; one or more nucleic acid molecules encoding a lasso cyclase; and one or more nucleic acid molecules encoding a RiPP recognition element (RRE)) are operably linked to a solubility enhancing polypeptide.

In an embodiment, a recombinantly produced, mature lasso peptide can be produced at a yield of about 0.5, 0.75, 1.0, 1.5, 2.0, 3.0, 4.0, 5.0 mg/L or more of culture media.

In an embodiment, a recombinantly produced mature lasso peptide has about 90, 95, 100, 105, or 110%, of the biological activity of a corresponding natural or wild-type lasso peptide.

In an embodiment a recombinantly produced mature lasso peptide has about 90, 95, 100, 105, or 110%, of the proteolytic stability of a corresponding natural or wild-type lasso peptide In an embodiment a recombinantly produced mature lasso peptide has about 90, 95, 100, 105, or 110%, of the thermal stability of a corresponding natural or wild-type lasso peptide.

In an embodiment a recombinantly produced mature lasso peptide has about 90, 95, 100, 105, or 110%, of the solubility of a corresponding natural or wild-type lasso peptide.

In an embodiment the lasso peptides are from an actinomycete, thermophile, or mesophile, and the host cell is a bacterium from the order Enterobacteriales, such as Escherichia coli. In an embodiment the host cell is a bacterium from the family Enterobacteriaceae. In an embodiment, the host cell is from the Firmicutes phylum, including for example, Bacillus sp., or Bacillus subtilis. In an embodiment, the host cell is a Sinorhizobium sp., such as Sinorhizobium meliloti, Sinorhizobium medicae, and Sinorhizobium fredii.

Mature lasso peptides, lasso precursor peptides, lasso leader peptidases, lasso cyclases, and RiPP recognition element can be actinomycete, thermophile, or mesophile peptides. This means that the mature lasso peptide, precursor peptide, lasso leader peptidase, lasso cyclase, or RiPP recognition element has about 90, 91, 92, 93, 94, 95, 96, 97, 98, 99, or 100% homology to a naturally occurring actinomycete, thermophile, or mesophile mature lasso peptide, lasso precursor peptide, lasso leader peptidase, lasso cyclase, or RiPP recognition element. In an embodiment a mature lasso peptide, precursor peptide, lasso leader peptidase, lasso cyclase, or RiPP recognition element has about 50, 60, 70, 80, 90% or more homology to a naturally occurring actinomycete, thermophile, or mesophile mature lasso peptide, lasso precursor peptide, lasso leader peptidase, lasso cyclase, or RiPP recognition element. In an embodiment a mature lasso peptide, precursor peptide, lasso leader peptidase, lasso cyclase, or RiPP recognition element can be a synthetic or a mutant lasso polypeptide.

Nucleic acid molecules that encode lasso precursor peptides, lasso leader peptidases, lasso cyclases, and RiPP recognition element can be actinomycete, thermophile, or mesophile nucleic acid molecules. This means that nucleic acid molecules encoding the lasso precursor peptides, lasso leader peptidase, lasso cyclase, or RiPP recognition element have about 90, 91, 92, 93, 94, 95, 96, 97, 98, 99, or 100% homology to naturally occurring actinomycete, thermophile, or mesophile nucleic acid molecules that encode lasso precursor peptides, lasso leader peptidases, lasso cyclases, or RiPP recognition elements. In an embodiment nucleic acid molecules that encode precursor peptides, lasso leader peptidases, lasso cyclases, or RiPP recognition elements have about 50, 60, 70, 80, 90% or more homology to a naturally occurring actinomycete, thermophile, or mesophile nucleic acid molecules that encode lasso precursor peptides, lasso leader peptidases, lasso cyclases, or RiPP recognition elements. In an embodiment nucleic acid molecules encoding a precursor peptide, lasso leader peptidase, lasso cyclase, or RiPP recognition element can be a synthetic nucleic acid or a mutant lasso nucleic acid.

Actinomycetes are members of the phylum Actinobacteria. Actinomycetes include, for example, Actinomycetaceae, Actinopolysporaceae, Catenulisporaceae, Actinospicaceae, Corynebacteriaceae, Dietziaceae, Gordoniaceae, Mycobacteriaceae, Nocardiaceae, Segniliparaceae, Tsukamurellaceae, Acidothermaceae, Cryptosporangiaceae, Frankiaceae, Geodermatophilaceae, Nakamurellaceae, Sporichthyaceae, Glycomycetaceae, Jiangellaceae, Kineosporiaceae, Beutenbergiaceae, Bogoriellaceae, Brevibacteriaceae, Cellulomonadaceae, Demequinaceae, Dermabacteraceae, Dermatophilaceae, Dermacoccaceae, Intrasporangiaceae, Jonesiaceae, Microbacteriaceae, Micrococcaceae, Promicromonosporaceae, Rarobacteraceae, Ruaniaceae, Sanguibacteraceae, Micromonosporaceae, Nocardioidaceae, Propionibacteriaceae, Pseudonocardiaceae, Streptomycetaceae, Nocardiopsaceae, Streptosporangiaceae, and Thermomonosporaceae.

Actinomycete lasso peptides include but are not limited to, for example, fusilassin from Thermobifida fusca; sphaericin from Planomonospora sphaedca; streptomonomicin from Streptomonospora alba; raynimysin from Streptomyces; gelsomycin from Streptomyces; adanomysin from Streptomyces; anantin from Streptomyces; frankimysin from Frankia; glycimysin from Actinomyces; AnantinB₁ from Streptomyces sp. NRRL-S-146; AnantinB₂ from Streptomyces sp. NRRL-S-146; Anantin C from Streptomyces olindensis; citrulassin A from Streptomyces aurantiacus NRRL B-3066; citrulassin B from Streptomyces aurantiacus NRRL B-2806; citrulassin C from Streptomyces sp. NRRL S-146; citrulassin D from Streptomyces katrae NRRL B-16271; citrulassin E from Streptomyces glaucescens NRRL 1SP-5155, Streptomyces glaucescens NRRL B-2899, Streptomyces glaucescens NRRL B-11408, or Streptomyces glaucescens NRRL B-2706; Citrulassin F from Streptomyces avermitilis NRRL B-16169; Keywimysin from Streptomyces sp. NRRL F-5702, Streptomyces sp. NRRL F-5681, or Streptomyces sp. NRRL F-2202; lagmysin form Streptomyces sp. NRRL S-118; LP2006 from Nocardiopsis alba; moomysin from Streptomyces cattleya sp. NRRL 8057; anantin B form Streptomyces sp. NRRL S-146; anantin C from Streptomyces olindensis, citrulassin A from Streptomyces albulus NRRL B-3066; citrulassin B from Streptomyces aurantiacus NRRL B-2806; citrulassin C from Streptomyces sp. NRRL 5-146; citrulassin D from Streptomyces katrae NRRL B-16271; citrulassin E from Streptomyces glaucescens NRRL 1SP-5155; citrulassin F from Streptomyces avermitilis NRRL B-16169; keywimysin from Streptomyces sp. NRRL; F-5681; keywimysin from Streptomyces sp. NRRLF-2202; keywimysin Streptomyces sp. NRRL F-5702; lagmysin Streptomyces sp. NRRL S-118; LP2006 from Nocardiopsis alba DSM 43377; moomysin from Streptomyces cattleya NRRL 8057; BI-32169-type from Streptomyces sp. AmelKG-E11A; RES-701-type from Streptomyces auratus; siamycin III from Streptomyces griseorubens; siamycin I from Streptomyces nodosus; propeptin from Microbispora rosea.

Thermophiles can be fungi, archaea, or bacteria that have an optimum growth temperature of about 50° or more, a maximum of up to about 70° C. or more, and a minimum of about 20° C. Thermophilic fungi include, for example, Acremonium, Aureobasidium, Cryptococcus, Filibasidium, Fusarium, Humicola, Magnaporthe, Mucor, Myceliophthora, Neurospora, Paecilomyces, Penicillium, Schizophyllum, Talaromyces, Thermoascus, Thielavia, and Tolypocladium. Thermophilic microorganisms are eubacteria or archaebacteria and include for example the following genera: Thermus, Bacillus, Thermococcus, Pyrococcus, Aeropyrum, Aquifex, Sulfolobus, Pyrolobus, or Methanopyrus. Specific examples include Thermobispora bispora, Thermobacculum terrenum, Thermobifida fusca, Thermobifida cellulosilytica, Thermoactinomyces vulgaris, Thermus aquaticus, Thermus thermophiles, Bacillus stearothermophilus, Aquifex pyrophilus, Geothermobacterium ferrireducens, Thermotoga maritime, Thermotoga neopolitana, Thermotoga petrophila, Thermotoga naphthophila, Acidianus infernus, Aeropyrum pernix, Archaeoglobus fulgidus, Archaeoglobus profundus, Caldivirga maquilingensis, Chlorofiexus aurantiacus, Desulfurococcus amylolyticus, Desulfurococcus mobilis, Desulfurococcus mucosus, Ferroglobus placidus, Geoglobus ahangari, Hyperthermus butylicus, Ignicoccus islandicus, Ignicoccus pacificus, Methanococcus jannaschii, Methanococcus fervens, Methanococcus igneus, Methanococcus infernus, Methanopyrus kandleri, Methanothermus fervidus, Methanothermus sociabilis, Palaeococcus ferrophilus, Pyrobaculum aerophilum, Pyrobaculum calidifontis, Pyrobaculum islandicum, Pyrobaculum oguniense, Pyrococcus furiosus, Pyrococcus abyssi, Pyrococcus horikoshii, Pyrococcus woesei, Pyrodictium abyssi, Pyrodictium brockii, Pyrodictium occultum, Pyrolobus fumarii, Staphylothermus marinus, Stetteria hydrogenophila, Sulfolobus solfataricus, Sulfolobus shibatae, Sulfolobus tokodaii, Sulfophobococcus zilligii, Sulfurisphaera ohwakuensis, Thermococcus kodakaraensis, Thermococcus celer, Thermococcus litoralis, Thermodiscus maritimus, Thermofilum pendens, Thermopmteus tenax, Thermoproteus neutrophilus, Thermosphaera aggregans, Vulcanisaeta distributa, and Vulcanisaeta souniana.

Mesophiles are microorganisms that grows best in moderate temperatures between about 20 and 50° C. Mesophiles can be archaea, bacteria or fungi.

### Methods of Recombinant Production of Peptides of Lasso Biosynthetic Gene Cluster

In an embodiment a mesophile, thermophile, or actinomycete lasso leader peptidase, lasso cyclase, lasso precursor peptide, lasso RiPP recognition element, or combination thereof can be produced. A host cell can be transformed with (1) a plasmid comprising a nucleic acid molecule encoding a mesophile, thermophile, or actinomycete lasso leader peptidase optionally operably linked to a solubility enhancing polypeptide; (2) a plasmid comprising a nucleic acid molecule encoding a mesophile, thermophile, or actinomycete lasso cyclase optionally operably linked to a solubility enhancing polypeptide; (3) a plasmid comprising a nucleic acid molecule encoding a mesophile, thermophile, or actinomycete lasso precursor peptide optionally operably linked to a solubility enhancing polypeptide; (4) a plasmid comprising a nucleic acid molecule encoding a mesophile, thermophile, or actinomycete lasso RiPP recognition element optionally operably linked a solubility enhancing polypeptide; or (5) combinations thereof, to generate a transformed host cell. The transformed host cell can be cultured in a culture medium. The lasso leader peptidase, lasso cyclase, lasso precursor peptide, lasso RiPP recognition element, or combination thereof can be extracted from the host cell or the culture media.

In an embodiment, lasso leader peptidase, lasso cyclase, lasso precursor peptide, or lasso RiPP recognition element can be produced at a yield of about 0.5, 0.75, 1.0, 1.5, 2.0, 3.0, 4.0, 5.0 mg/L of culture media.

In an embodiment, a solubility enhancing peptide can be cleaved from a lasso leader peptidase, lasso cyclase, lasso precursor peptide, or lasso RiPP recognition element using a protease.

In an embodiment, a recombinantly produced lasso leader peptidase, lasso cyclase, lasso precursor peptide, lasso RiPP recognition element has about 90, 95, 100, 105, 110%, or more biological activity of a corresponding natural or wild-type lasso leader peptidase, lasso cyclase, lasso precursor peptide, or lasso RiPP recognition element.

In an embodiment a recombinantly produced lasso leader peptidase, lasso cyclase, lasso precursor peptide, lasso RiPP recognition element has about 90, 95, 100, 105, or 110%, of the proteolytic stability of a corresponding natural or wild-type lasso leader peptidase, lasso cyclase, lasso precursor peptide, or lasso RiPP recognition element.

In an embodiment a recombinantly produced lasso leader peptidase, lasso cyclase, lasso precursor peptide, lasso RiPP recognition element has about 90, 95, 100, 105, or 110%, of the thermal stability of a corresponding natural or wild-type lasso leader peptidase, lasso cyclase, lasso precursor peptide, or lasso RiPP recognition element.

In an embodiment a recombinantly produced lasso leader peptidase, lasso cyclase, lasso precursor peptide, lasso RiPP recognition element has about 90, 95, 100, 105, or 110%, of the solubility of a corresponding natural or wild-type lasso leader peptidase, lasso cyclase, lasso precursor peptide, lasso RiPP recognition element.

In an embodiment, a recombinantly produced lasso RiPP recognition element can bind a leader of a lasso precursor peptide with an affinity for a lasso leader peptide of about 30, 40, 50, 60, 70, 80, or more nM.

### Methods of In Vitro Reconstitution of Mature Lasso Peptides

Mature lasso peptides can be reconstituted in vitro (also called total biosynthesis). In an embodiment, one or more purified recombinantly produced lasso leader peptidases, one or more purified recombinantly produced lasso cyclases, one or more purified recombinantly produced lasso precursor peptides, and one or more purified recombinantly produced lasso RiPP recognition elements are combined in vitro under suitable conditions to produce a lasso peptide. A lasso peptide can be produced at a yield of about 0.5, 1, 2, 3, 4, 5, 6 mg/L or more.

In an embodiment, a lasso peptide can be produced in vitro by combining one or more purified recombinantly produced lasso precursor peptides, one or more purified recombinantly produced lasso cyclases, and adenosine triphosphate (ATP) in vitro under conditions suitable for lasso peptide formation, such that a mature lasso peptide is produced.

In an embodiment, a mature lasso peptide can be produced in vitro by combining one or more purified recombinant lasso precursor peptides lacking a leader, one or more recombinantly produced lasso cyclases, and adenosine triphosphate (ATP) in vitro under conditions suitable for lasso peptide formation, such that a mature lasso peptide is produced. A lasso precursor peptide is comprised of a leader and a core. For this method, the leader portion of the lasso precursor peptide is not necessary. The lasso precursor peptide can be cleaved (using e.g., a protease) such that the leader is removed and the core portion used in the method. Alternatively, a lasso precursor peptide lacking a leader can be produced recombinantly by transforming a host cell with a polynucleotide encoding only the core portion of a lasso precursor peptide. This core peptide can then be purified and used in the method. In an embodiment a lasso precursor peptide lacking a leader portion is missing about 8, 10, 12, 15, 20, 30 or more amino acids of the leader portion. In an embodiment a lasso precursor peptide lacking a leader portion is missing all of the leader portion amino acids. A lasso peptide can be produced at a yield of about 0.5, 1, 2, 3, 4, 5, 6 mg/L or more.

The core sequences of lasso precursor peptides are known to those of skill in the art, and can be found in, for example Tietz et at, Nat. Chem. Biol. 13:470 (2017), see supplementary Table 8.

In vitro reconstituted mature lasso peptides can have about 50, 60, 70, 80, 90, 100, 110%, or more biological activity as compared to a natural or wild-type lasso peptide.

In vitro reconstituted mature lasso peptides can have about 50, 60, 70, 80, 90, 100, 110%, or more proteolytic stability as compared to a natural or wild-type lasso peptide In vitro reconstituted mature lasso peptides can have about 50, 60, 70, 80, 90, 100, 110%, or more thermal stability as compared to a natural or wild-type lasso peptide.

In vitro reconstituted mature lasso peptides can have about 50, 60, 70, 80, 90, 100, 110%, or more solubility as compared to a natural or wild-type lasso peptide.

In an embodiment the in vitro reconstituted mature lasso peptides are derived from an actinomycete or thermophile and the host cell is a bacterium from the order Enterobacteriales, such as Escherichia coli. In an embodiment the host cell is a bacterium from the family Enterobacteriaceae.

### Methods of Screening

An embodiment provides methods of screening for activity of a lasso peptide. One or more lasso precursor peptides can be displayed in a display library. In an embodiment the lasso precursor peptide can be displayed as a fusion to a protein tag, such as Aga2p. See Bader & Wittrup, Nat. Biotechnol. 1997; 15:553. The lasso precursor peptide is displayed on the surface of library, by for example the C terminus. This is a lasso precursor peptide display library. The lasso precursor peptide display library can be contacted with one or more of a purified lasso cyclase, a purified lasso leader peptidase, and a purified lasso RiPP recognition element to form a lasso peptide display library. That is, after contacting the lasso precursor peptide display library with one or more of a purified lasso cyclase, a purified lasso leader peptidase, and a purified lasso RiPP recognition element, mature lasso peptides are displayed on the surface of the display library to form a mature lasso peptide display library. The enzymes and proteins used in this method can be recombinantly produced.

It is important that the purified lasso cyclase, lasso leader peptidase, and/or lasso RiPP recognition element are stable and have robust activity in order to form the mature lasso peptide display library. The purified lasso cyclase, lasso leader peptidase, and/or lasso RiPP recognition element can be recombinant peptides produced by methods described herein. The lasso cyclase, lasso leader peptidase, and/or lasso RiPP recognition element can be operably linked to a solubility enhancing polypeptide.

A lasso precursor peptide display library or mature lasso peptide display library can comprise a phage display library. A phage display library can be a collection of phage that have been genetically engineered to express one or more lasso precursor peptides on their outer surface. In an embodiment nucleic acid molecules encoding the lasso precursor peptides are inserted in frame into a gene encoding a phage capsule protein. In another embodiment, a phage display library is a collection of phage that display one or more mature lasso peptides on their outer surface.

A display library can be, for example, a phage display library, a phagemid display library, a virus display library, a bacterial cell display library, yeast display library, a λgt11 library, an in vitro library selection system (CIS display), an in vitro compartmentalization library, an antibody-ribosome-mRNA (ARM) ribosome display library, or a ribosome display library.

Methods of making and screening such display libraries are well known to those of skill in the art and described in, e.g., Molek et al, (2011) Molecules 16, 857-887; Boder et al., (1997) Nat Biotechnol 15, 553-557; Scott et al, (1990) Science 249, 386-390; Brisette et al. (2007) Methods Mol Biol 383, 203-213; Kenrick et al. (2010) Protein Eng Des Sel 23, 9-17; Freudl et al, (1986) J Mol Biol 188, 491-494; Getz et al. (2012) Methods Enzymol 503, 75-97; Smith et al. (2014) Curr Drug Discov Technol 11, 48-55; Hanes, et al. (1997) Proc Natl Acad Sci USA 94, 4937-4942; Lipovsek et al., (2004) J Imm Methods 290, 51-67; Ullman et al (2011) Brief. Funct. Genomics, 10, 125-134; Odegrip et al. (2004) Proc Natl Acad Sci USA 101, 2806-2810; and Miller et al. (2006) Nat Methods 3, 561-570.

A mature lasso peptide display library can be screened for biological activity such as anti-microbial activity; receptor antagonist activity (e.g., endothelin receptor, natriuretic system, glucagon receptor), enzyme inhibitor activity (e.g., inhibitor of smooth muscle myosin light chain kinase (MLCK), prolyloligopeptidase, RNA polymerase), and inhibitor activity of, for example, HIV. Therefore, test agents such as bacteria, fungi, viruses, prokaryotic or eukaryotic cells, drugs, cellular receptor proteins, proteins, nucleic acids, enzymes, and small molecules can be added to a mature lasso peptide display library. The mature lasso peptides can be mesophile mature lasso peptides, actinomycetes mature lasso peptides, or thermophile mature lasso peptides.

The lasso peptide display library can then be contacted with one or more test agents. The effect of the lasso peptides on the test agent can then be determined. Lasso peptides can have a wide variety of biological activities including, for example, inhibition of binding, inhibition of enzyme activity, bactericidal activity, bacteriostatic activity, virucidal activity, and virustatic activity.

In an embodiment, a screening step can also serve as the step of recovering a test agent that binds to the mature lasso peptide.

Methods well known to those skilled in the art can be applied to screening of display libraries. Examples include a solid-phase screening methods and liquid-phase screening methods. Solid-phase screening methods can involve, for example, immobilizing target agents onto a solid phase, and contacting lasso peptides contained in a liquid phase with the target agents, removing unbound lasso peptides nonspecifically bound lasso peptides, and then selectively separating lasso peptides bound with the target agent to screen for a peptide having, for example, a desired binding activity. A liquid-phase screening method can involve, for example, contacting lasso peptides with target agents in a solution, removing unbound lasso peptides and nonspecifically bound lasso peptides, and then selectively separating the lasso peptides bound with target agents.

### Definitions

A recombinant host cell, transgenic host cell, or transformed host cell is a cell into which one or more foreign or exogenous nucleic acid molecules, synthetic nucleic acid molecules, or plasmids have been introduced or inserted into the cell. The one or more foreign nucleic acid molecules, synthetic nucleic acid molecules, or plasmids do not occur in the host cell in nature. An exogenous or foreign nucleic acid molecule can be from a different species (and so heterologous), or from the same species (and so homologous), relative to the cell being transformed. An exogenous gene can include a homologous gene that occupies a different location in the genome of the cell or is under different control, relative to the endogenous copy of the gene. An exogenous gene may be present in more than one copy in the cell. An exogenous gene can be maintained in a cell as an insertion into the genome or as an extrachromosomal molecule.

Suitable host cells for expression of nucleic acid molecules are microbial cells that can be found broadly within the fungal or bacterial families and that grow over a wide range of temperature, pH values, and solvent tolerances. Examples of suitable host strains include but are not limited to fungal or yeast species, such as Aspergillus, Trichoderma, Saccharomyces, Pichia, Candida, Hansenula, and Kluyveromyces, or bacterial species, such as member of the proteobacteria and actinomycetes as well as the specific genera Acinetobacter, Arthrobacter, Brevibacterium, Acidovorax, Bacillus, Clostridia, Streptomyces, Escherichia (e.g., E. coli), Salmonella, Pseudomonas, and Cornyebacterium. A host cell can be a mesophile, thermophile, or actinomycete cell.

Transformation is a process of introducing foreign or exogenous genetic material into a host cell.

An isolated nucleic acid molecule, peptide, or polypeptide, refers to nucleic acid molecule, peptide, or polypeptide that is separated from at least one contaminant with which it is ordinarily associated in its source. Thus, an isolated nucleic acid molecule, isolated peptide, or isolated polypeptide is present in a form or setting that is different from that in which it is found in nature. An isolated nucleic acid molecule can be a naturally-occurring polynucleotide that is not immediately contiguous with one or both of the 5′ and 3′ flanking genomic sequences that it is naturally associated with.

A nucleic acid molecule or polynucleotide is a nucleic acid molecule (e.g., DNA or RNA) that can comprise coding sequences necessary for the production of a peptide, polypeptide, or protein precursor. The encoded polypeptide may be a full-length polypeptide, a fragment thereof (less than full-length), or a fusion of either the full-length polypeptide or fragment thereof with another polypeptide, yielding a fusion polypeptide. A peptide, protein, or polypeptide is any chain of amino acids, regardless of length or post-translational modification (e.g., glycosylation or phosphorylation).

An expression vector, plasmid, or recombinant DNA construct is a vehicle for introducing one or more nucleic acid molecules into a host cell. The nucleic acid molecule can be one that has been generated via human intervention, including by recombinant means or direct chemical synthesis. The nucleic acid molecule can include one or more nucleic acid elements that permit transcription and/or translation of a particular nucleic acid molecule. An expression vector can be part of a plasmid, virus, or nucleic acid fragment, or other suitable vehicle. An expression vector can include, for example, a nucleic acid to be transcribed operably linked to a promoter.

Operably linked means two or more nucleic acid molecules that are functionally linked together, such as one or more control sequences (e.g., a promoter) and one or more target nucleic acid molecules (e.g., molecules that encodes a protein) or two or more target nucleic acid molecules that are linked. Where two or more target nucleic acid molecules are linked the result can be a fusion protein. In an example, a promoter is operably linked to a target nucleic acid molecule where it can mediate transcription of the target nucleic acid molecule.

A purified polypeptide or peptide is a polypeptide or peptide preparation that is substantially free of cellular material, other types of polypeptides, peptides, chemical precursors, chemicals used in synthesis of the polypeptide or peptide, or combinations thereof. A polypeptide or peptide preparation that is substantially free of cellular material, culture medium, chemical precursors, chemicals used in synthesis of the polypeptide or peptide, etc., has less than about 30%, 20%, 10%, 5%, 1% or more of other polypeptides or peptides, culture medium, chemical precursors, and/or other chemicals used in synthesis. Therefore, a purified polypeptide or peptide is about 70%, 80%, 90%, 95%, 99% or more pure. A purified polypeptide or peptide does not include unpurified or semi-purified cell extracts or mixtures of polypeptides that are less than 70% pure.

All patents, patent applications, and other scientific or technical writings referred to anywhere herein are incorporated by reference herein in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### Examples

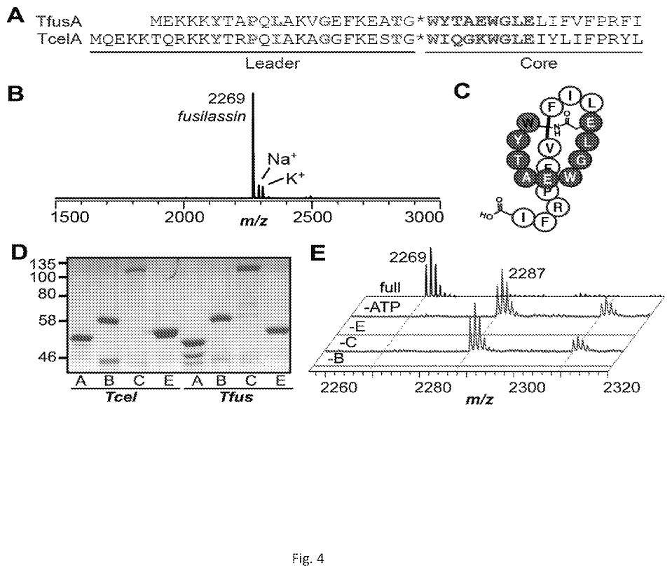

Lasso peptide biosynthetic gene clusters from thermophiles were identified in an attempt to provide more stable and soluble lasso enzymes. Biosynthetic gene clusters from Thermobispora bispora, Thermobifida fusca (“Tfus”), and Thermobifida cellulosilytica (“Tcel”) were identified as targets. An unusual feature of the lasso peptides of Tfus and Tcel is that they harbor the largest proteinogenic amino acid, Trp, as the first core residue (FIG. 4A). Previous reports incorrectly assumed that only small residues (Gly, Ala, Ser, Cys) were biosynthetically compatible with lasso peptide formation. Notably, RODEO has identified cases where every residue except Pro is represented as the first residue of the core. Indeed, two cases were identified where Leu resides at this position (FIG. 3C).

To validate Tfus/Tcel as true lasso peptide BGCs, TfusA was cloned into a pET28 derivative that provides an N-terminal maltose-binding protein (MBP) tag. A second “Duet” plasmid harbored tfusC and tfusE-B. Upon E. coli expression, “fusilassin” was detected by MALDI-TOF-MS (m/z 2269, FIG. 4B) after a methanolic extraction. After HPLC, high-resolution Orbitrap MS/MS analysis confirmed the identity of the peptide. It appears that the peptide is threaded, as opposed to a “branched cyclic” conformation, based on the following data: (i) resistance of centrally located amide NHs to deuterium exchange, (ii) thermal stability by HPLC after heat treatment (95° C., 4 h), and (ill) resistance to protease digestion. Anecdotal evidence also supports the threaded state given that no lasso peptide has ever been isolated in the branched cyclic form.

Fusilassin lasso sequences for Thermoactinomyces vulgaris and Thermobifida fusca YX were determined using RODEO. See Tietz et al. A leader for Thermoactinomyces vulgaris was determined to be MEKQKETKKEYSSPRLIELGDIVEITF (SEQ ID NO:10) and the core was determined to be GGKPGWGSDTYSQRYPRSDED (SEQ ID NO:11). A leader for Thermobifida fusca YX was determined to be MEKKKYTAPQLAKVGEFKEATG (SEQ ID NO:12) and the core was determined to be WYTAEWGLELIFVFPRFI (SEQ ID NO:13).

The proteins from Thermobifida fusca were cloned and obtained as full length and as a soluble protein so this gene cluster was targeted for heterologous expression in E. coli.

The pACYCDuet plasmid was used as the vector backbone for the biosynthetic gene cluster. Gibson assembly was used to clone 5′-TfusE-RBS-TfusB-3′ into MCS2 (primers: TfusB_F/R and TfusB_F/R). A ribosome binding site was inserted between the genes by adding the appropriate sequence into TfusE_R and TfusB_F. TfusC was then cloned into MCS1 using restriction enzyme digestion (primers: TfusC_F/R). See Table 1.

TABLE 1

| Name | Primer (5′-3′) |
|---|---|
| TfusE_F | ATGGCAGATCTCAATTGGATATCAATGG |
| (SEQ ID NO: 14) | AGACAACAGGAGCTGAATTCCG |
| TfusE_R | GCTCATATGTATATCTCCTTCTTATACT |
| (SEQ ID NO: 15) | TAACTAATCATGGCAGCGCCATCCCG |
| TfusB_F | TTAGTTAAGTATAAGAAGGAGATATACA |
| (SEQ ID NO: 16) | TATGAGCGAGAACGTAGTGCTGC |
| TfusB_R | TTTACCAGACTCGAGGGTACCTCAGTCC |
| (SEQ ID NO: 17) | ACGGTGATCAGACGGC |
| TfusC_F | AAAAGTCGACATGGTCGGTTGCATCAGT |
| (SEQ ID NO: 18) | CCTTAC |
| TfusC_R | AAAGCGGCCGCTCAGCTCCTGTTGTCTC |
| (SEQ ID NO: 19) | CACCG |

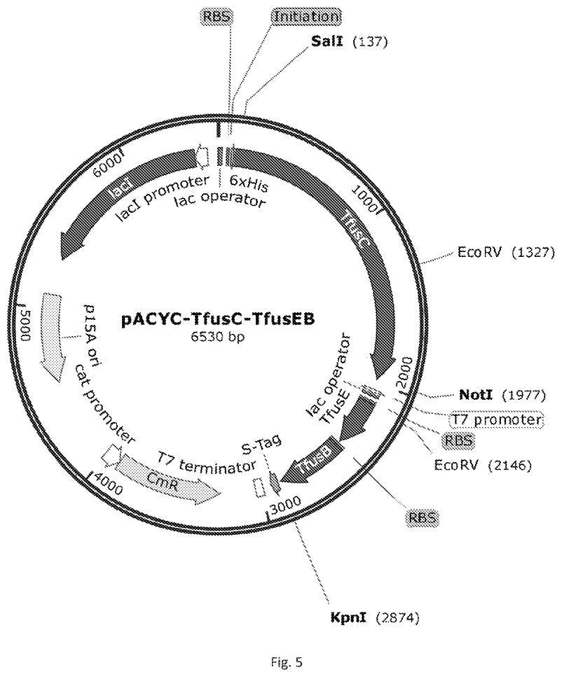

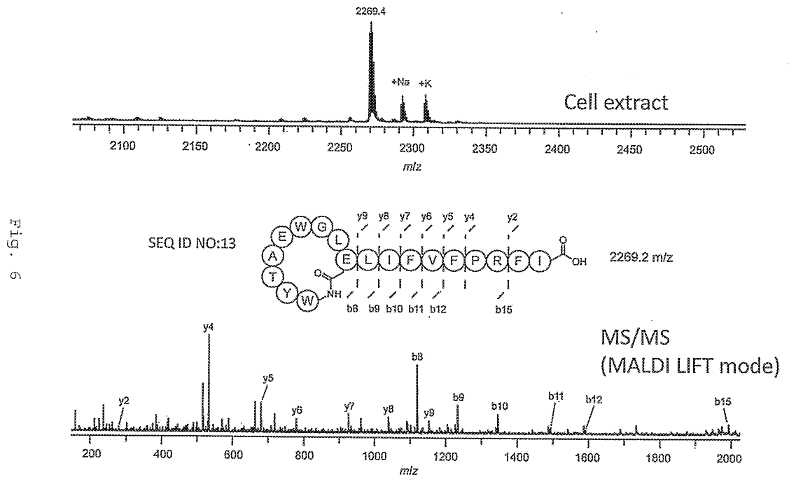

The precursor peptide was cloned into a modified pET28 plasmid in frame with an N-terminal MBP tag. See FIG. 5. The pET28 and pACYC plasmids were co-transformed into E. coli, which was grown to 0.8 OD₆₀₀. The plasmids were induced with 0.5 mM IPTG for 18 hours at 37° C. Cell pellets were harvested and extracted with methanol. The fusilassin lasso peptide was analyzed by MALDI-TOF-MS. The MALDI-TOF-MS results are shown in FIG. 6. Table 2 shows the protein name, molecular weight, and concentration of the protein purified from E. coli heterologous expression

TABLE 2

| | MW | Conc. | Conc. |
|---|---|---|---|
| | (Da) | (mg/mL) | (μM) |
| TfusA | 50400 | 5.24 | 104 |
| TfusB | 62600 | 3.4 | 54 |
| TfusE | 55800 | 3.5 | 63 |
| TfusC | 113300 | 1.71 | 15 |
| TfusCdM1-R58 | 107200 | 19.7 | 184 |
| TfusCdM1-V191 | 92600 | 3.48 | 38 |
| TcelA | 51200 | 9.22 | 180 |
| TcelB | 61200 | 7.06 | 115 |
| TcelE | 55700 | 0.84 | 15 |
| TcelC | 112500 | 1.79 | 16 |

This represents the first time an actinomycete-derived lasso peptide has successfully been expressed in E. coli.

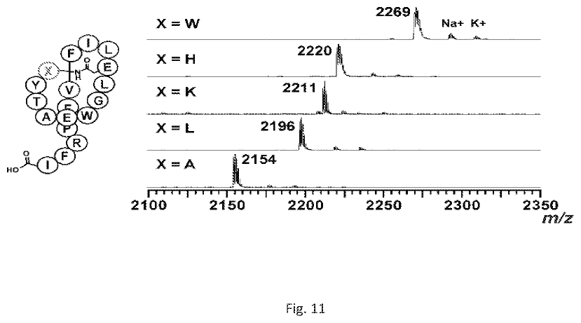

The two plasmid method of production of lasso peptides was used to evaluate the biosynthetic tolerance of the fusilassin pathway (FIG. 11). In all other studied cases of lasso peptides, the first position of the core peptide was largely intolerant to substitution. The two plasmid methodology was used to explore a subset of chemical space at position one of fusilassin. Substitution of the first position (Trp) of the fusilassin core peptide with Phe, Tyr, Lys, His, Ala and Leu resulted in cyclized fusilassin. See FIG. 11.

Fusilassin in vitro reconstitution. With Tfus confirmed, each protein necessary to carry out the in vitro biosynthetic reconstitution of fusilassin was cloned, expressed as MBP fusions, and purified from E. coli (FIG. 4D-E). FIG. 4E shows that full enzymatic processing occurs in the presence of all enzymes and ATP.

Table 3 shows the reaction conditions for reconstituting the Tfus and Tcel lasso peptide biosynthetic systems. A is the precursor peptide, B is the leader peptidase, C is the lasso cyclase; E is the RRE (RiPP) Recognition Element. The reactions were run for about 18 hours at room temperature.

TABLE 3
Synthetase Reactions

| | A | C | E | B | TEV | Buffer | Water | Total |
|---|---|---|---|---|---|---|---|---|
| | (25 μM) | (2 μm) | (2 μM) | (2 μM) | (μL) | (μL) | (μL) | (μL) |
| TfusACEB | 8.7 | 8.0 | 1.91 | 2.2 | 3.0 | 6.0 | 30.3 | 60.0 |
| TcelACEB | 5.0 | 7.5 | 8.0 | 1.0 | 3.0 | 6.0 | 29.5 | 60.0 |

These data represent the first lasso peptide system where each constituent protein is individually expressed and purified at mg/L levels while also exhibiting robust in vitro activity. The production of a high-yielding, functional lasso cyclase is notable as this has been the principal obstacle to successful in vitro lasso peptide production. Lasso cyclase can be produced at greater than 20 mg/L of culture using the methods described herein. TfusE, TfusB, and TfusA have been expressed as MBP fusions in quantities from about 20 to about 40 or more mg/L culture using the methods described herein.

Fusilassin has been reconstituted via in vitro biosynthesis at <mol % lasso cyclase, and at lasso peptide precursor amounts of up to 1 mmol. Fusilassin has been produced at multi-milligram amounts using only the purified enzymes described above.

The RiPP Recognition Element (RRE) is a domain of ˜90 aa with structural homology to PqqD. Ubiquitous to lasso peptide BGCs (FIGS. 1B, 3B), RREs are widely deployed in prokaryotic RiPP BGCs and govern the association between the leader peptide and biosynthetic protein(s). RRE:leader binding and most dissociation constants are in the nM range. A few RRE structures are available with each adopting a triple α-helix/β-sheet fold. Interestingly, the leader peptide binds as if it were the fourth α-sheet.

Additional Constructs. Other constructs for expression include using codon optimized lasso peptide nucleic acids. For example, nucleic acid molecules encoding a lasso precursor peptide, a solubility enhancing polypeptide, a lasso leader peptidase; a lasso cyclase; and a RiPP recognition element can be host cell “codon optimized”, i.e., at least 50%, 60%, 70%, 75%, 80%, 85%, 90% of 95% of the codons of the nucleic acids are replaced with codons that encode the same amino acid but that are preferred by the host cells, e.g., E. coli cells, thus improving or optimizing expression in the E. coli cells. To codon optimize the nucleic acid molecules, a nucleic acid molecule is generated that alters “wild-type” codons to codons more frequently utilized by the host cell (e.g., E. coli).

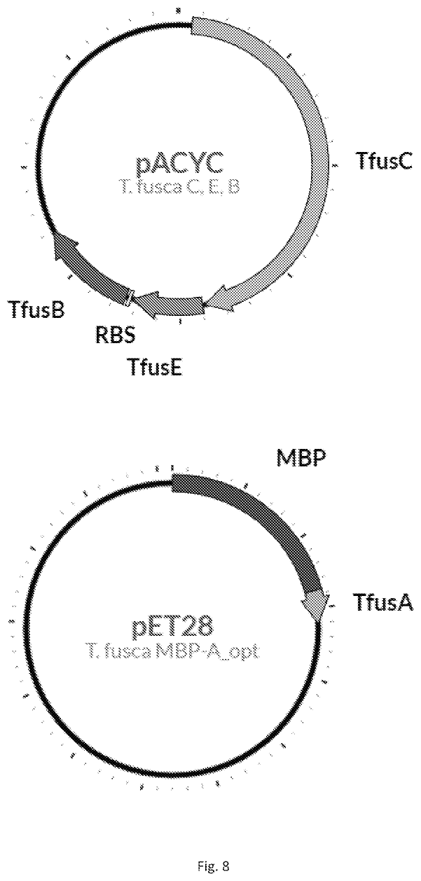

In an example, TfusA that has been codon optimized for E. coli is operably linked to a solubility enhancing peptide (e.g., MBP) on a first plasmid and TfusC, TfusE, and TfusB can be present on a second plasmid. See FIG. 8. Both of these plasmids are used to transform a host cell.

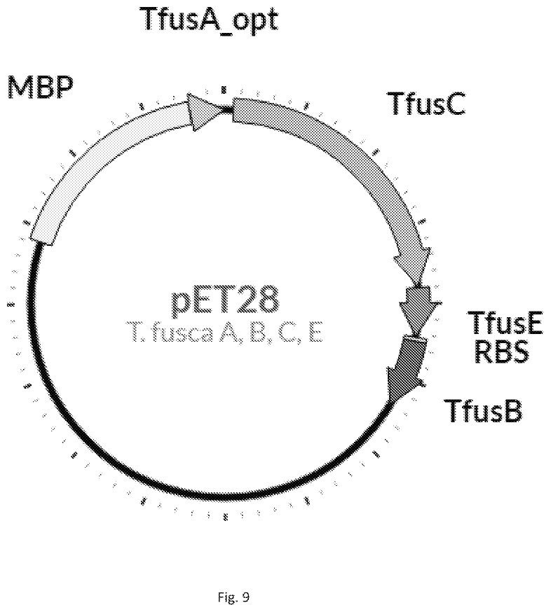

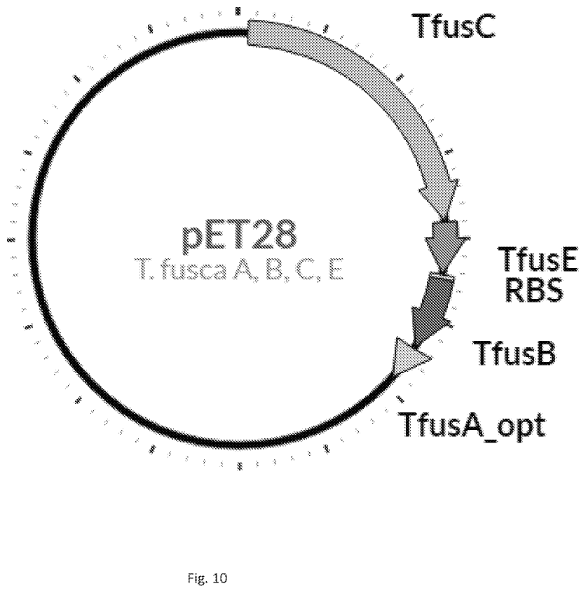

Other examples of a construct for expression of a lasso peptide are shown in FIGS. 9 and 10. A codon optimized lasso leader precursor (e.g. TfusA) is operably linked to a solubility enhancing peptide (e.g., MBP). Nucleic acid molecules encoding to a lasso leader peptidase; a lasso cyclase; and a RiPP recognition element (RRE) (e.g., TfusC, TfusE, and TfusB) are also present within the construct.

1. A method of producing a mature lasso peptide comprising:
(a) transforming a host cell with a first plasmid comprising a nucleic acid molecule encoding a lasso precursor peptide operably linked to a solubility enhancing polypeptide and a second plasmid comprising a nucleic acid molecule encoding a lasso leader peptidase; a nucleic acid molecule encoding a lasso cyclase; and a nucleic acid molecule encoding a RiPP recognition element (RRE) to generate a transformed host cell;
(b) culturing the transformed host ceil in a culture media;
(c) extracting the mature lasso peptide from the host cell or the culture media.

2. The method of claim 1, wherein the lasso peptide is produced at a yield of more than 0.5 mg/L of culture media.

3. The method of claim 1, wherein the nucleic acid molecule encoding a lasso precursor peptide is an actinomycete or thermophile nucleic acid molecule; the nucleic acid molecule encoding a lasso leader peptidase is an actinomycete or thermophile nucleic acid molecule; the nucleic acid molecule encoding a lasso cyclase is an actinomycete or thermophile nucleic acid molecule; the nucleic acid molecule encoding a RiPP recognition element (RRE) is an actinomycete or thermophile nucleic acid molecule; or combinations thereof.

4. The method of claim 3, wherein the host ceil is a mesophile.

5. The method of claim 1, wherein the mature lasso peptide is an actinomycete lasso peptide or a thermophile lasso peptide.

6. A method of producing one or more peptides of a lasso biosynthetic gene cluster comprising:
(a) transforming a host cell with:
(i) a plasmid comprising a nucleic acid molecule encoding an
actinomycete lasso cyclase or a thermophile lasso cyclase operably linked to a solubility enhancing polypeptide;
(ii) a plasmid comprising a nucleic acid molecule encoding a
thermophile lasso leader peptidase or an actinomycete lasso leader peptidase operably linked to a solubility enhancing polypeptide;
(iii) a plasmid comprising a nucleic acid molecule encoding a
thermophile lasso RiPP recognition element or an actinomycete lasso RiPP recognition element operably linked to a solubility enhancing polypeptide;
(iv) a plasmid comprising a nucleic acid molecule encoding a
thermophile lasso precursor peptide operably linked to a solubility enhancing polypeptide; or
(v) a combination thereof;
to generate a transformed host cell;
(b) culturing the transformed host ceil in a culture media;
(c) extracting the one or more peptides of a lasso biosynthetic gene cluster from the host cell or the culture media.

7. The method of claim 6, wherein the host ceil is a mesophile.

8. A method of producing a mature lasso peptide in vitro comprising combining one or more purified recombinant actinomycete or thermophile lasso cyclases, one or more purified recombinant actinomycete or thermophile lasso leader peptidases, one or more purified recombinant actinomycete or thermophile lasso RiPP recognition elements, and one or more purified recombinant actinomycete or thermophile lasso precursor peptides) in vitro under conditions suitable for lasso peptide formation, such that a mature lasso peptide is produced.

9. The method of claim 8, wherein the lasso peptide is produced at a yield of more than 1 mg/L.

10. (canceled)

11. (canceled)

12. (canceled)

13. (canceled)

14. (canceled)

15. (canceled)

16. (canceled)

17. (canceled)

18. (canceled)

19. The method of claim 1, wherein the mature lasso peptide has 90% or more biological activity of a corresponding wild-type lasso peptide.

20. The method of claim 6, wherein the one or more peptides of a lasso biosynthetic gene cluster have 90% or more biological activity of corresponding wild-type peptides of a lasso biosynthetic gene cluster.

21. The method of claim 8, wherein the mature lasso peptide has 90% or more biological activity of a corresponding wild-type lasso peptide.

22. The method of claim 1, wherein one or more of the nucleic acid molecule encoding a lasso precursor peptide operably linked to a solubility enhancing polypeptide, the nucleic acid molecule encoding a lasso leader peptidase, the nucleic acid molecule encoding a lasso cyclase; and the nucleic acid molecule encoding a RiPP recognition element (RRE) are codon optimized for the host cell.

23. The method of claim 6, wherein one or more of the nucleic acid molecule encoding an actinomycete lasso cyclase or a thermophile lasso cyclase operably linked to a solubility enhancing polypeptide; the nucleic acid molecule encoding a thermophile lasso leader peptidase or an actinomycete lasso leader peptidase operably linked to a solubility enhancing polypeptide; the nucleic acid molecule encoding a thermophile lasso RiPP recognition element or an actinomycete lasso RiPP recognition element operably linked to a solubility enhancing polypeptide; and the nucleic acid molecule encoding a thermophile lasso precursor peptide or a operably linked a solubility enhancing polypeptide are codon optimized for the host cell.

24. The method of claim 1, wherein the solubility enhancing polypeptide is operably linked to the N-terminus of the nucleic acid molecule encoding a lasso precursor peptide.

25. The method of claim 1, wherein the solubility enhancing polypeptide is operably linked to the C-terminus of the nucleic acid molecule encoding a lasso precursor peptide.
